(19) United States
(12) Patent Application Publication
(10) Pub. No.: US 2022/0160259 A1
(43) Pub. Date: May 26, 2022

(54) METHOD AND SYSTEM FOR DETECTION AND ANALYSIS OF THORACIC OUTLET SYNDROME (TOS)

(71) Applicant: Baylor College of Medicine, Houston, TX (US)

(72) Inventors: Bryan Burt, Houston, TX (US); Bijan Najafi, Houston, TX (US); Mohsen Zahiri, Houston, TX (US); Changhong Wang, Houston, TX (US)

(73) Assignee: Baylor College of Medicine, Houston, TX (US)

(21) Appl. No.: 17/594,076

(22) PCT Filed: Apr. 2, 2020
(86) PCT No.: PCT/US20/26473

§ 371 Date: Oct. 1, 2021

Related U.S. Application Data
(60) Provisional application No. 62/830,138, filed on Apr. 5, 2019.

(51) Int. Cl. A61B 5/11; A61B 5/00

(52) U.S. Cl. CPC A61B 5/1121; A61B 2562/0219; A61B 5/6824; A61B 5/7207

(57) ABSTRACT

Motion data collected by a sensing device attached to a patient's arm may be used to determine whether the arm is subject to thoracic outlet syndrome (TOS) Motion data regarding motion of an arm of a patient may be received from a sensing device. One or more extremity performance parameters for the arm may be determined based, at least in part, on the motion data. A determination may be made based, at least in part, on the one or more extremity performance parameters whether the arm is subject to TOS.

### CROSS-REFERENCE TO RELATED PATENT APPLICATIONS

This application claims the benefit of U.S. Provisional Patent Application No. 62/830,138 filed on Apr. 5, 2019 and entitled “METHOD AND SYSTEM FOR DETECTION AND ANALYSIS OF THORACIC OUTLET SYNDROME (TOS),” which is hereby incorporated by reference.

### FIELD OF THE DISCLOSURE

The instant disclosure relates to medical diagnostics and intervention. More specifically, certain portions of this disclosure relate to a computerized platform for evaluating thoracic outlet syndrome (TOS).

### BACKGROUND

Thoracic outlet syndrome (TOS) includes a trio of debilitating musculoskeletal disorders that result from compression of the neurovascular structures that serve the upper extremities. Neurogenic TOS (nTOS) includes compression of the brachial plexus which may result in debilitating pain in one or both upper extremities and, in some cases, paresthesias. Venous TOS (vTOS) includes subclavian vein thrombosis, upper extremity swelling, and cyanosis secondary to subclavian vein compression. Arterial TOS (aTOS) includes subclavian artery compression, which may lead to ischemia of upper extremities.

nTOS is the most common form of TOS, comprising more than 90% of reported TOS cases. nTOS may be accompanied by a constellation of symptoms including upper extremity pain and paresthesias, neck and shoulder pain, extremity weakness, Raynaud's syndrome, and occipital headaches. In nTOS, dynamic compression of the brachial plexus may occur in the passage of the brachial plexus through the scalene triangle, formed by the anterior and middle scalene muscles where they connect to the first rib. Narrowing of the scalene triangle may be caused by scalene muscle hypertrophy secondary to traumatic or repetitive motion injury, together with an anatomic abnormality of the first rib, such as a high-riding first rib or an extra cervical rib. Symptoms may vary as movement of extremities may affect the positioning of the scalene triangle but, over time, may progress into constant debilitating pain and paresthesias. Individuals with nTOS may be impeded in their ability to perform daily tasks and tasks required by their chosen occupations, especially individuals in occupations requiring a substantial amount of physical activity.

Estimates of instances of nTOS range from three to eighty cases per one-thousand in population. TOS is highly prevalent among industrial workers and athletes and is also prevalent among computer users and musicians that are evaluated for work-related pain. In spite of its prevalence, the quality of metrics for evaluating and managing patients with nTOS is lacking. For example, current methods for diagnosing and evaluating nTOS, and evaluating the efficacy of treatments for nTOS, are based almost entirely on subjective factors, that may be influenced by human bias and/or error. For example, only one percent of nTOS patients are diagnosed based on objective findings such as hand atrophy or electromyography (EMG). A cervical rib is present in up to 30% of individuals with nTOS, but is not itself diagnostic of nTOS as cervical ribs are present in approximately 1.1% of the population, far greater than the percentage of the population with nTOS.

Current methods for nTOS treatment typically begin with physical therapy and then proceed to surgery if pain becomes debilitating. nTOS is typically treated first with physical therapy to soften the scalene muscles and relieve brachial plexus compression. Other non-operative therapies, such as ergonomic modifications and pain management may also be used. However, when symptoms become debilitating, surgery including first rib resection and scalenectomy may be offered to anatomically decompress the thoracic outlet. The response to physical therapy is highly variable with between thirty-seven percent and eighty-eight percent of nTOS patients undergoing physical therapy ultimately requiring corrective surgery. In some patients, physical therapy may even exacerbate nTOS symptoms making the patients worse off than they would have been without participating in physical therapy. Current methods of nTOS diagnosis and evaluation are subjective and do not provide objective measures for determining whether a patient is likely to respond positively to physical therapy or surgery.

Furthermore, no objective tests exist to determine the efficacy of physical therapy or surgery in a patient after the patient undergoes treatment. For example, efficacy of therapy is frequently evaluated using questionnaires that may suffer from selection and scale perception biases inherent to self-reporting modalities. Common classifications for the efficacy of treatment include Derkash's classification, which includes a surgeon assessment of excellent, good, fair, or poor results. Other methods of treatment evaluation, such as the disabilities of the arm, shoulder and hand (DASH) questionnaire, the cervical-brachial symptom questionnaire (CSBQ), and the short-form 12 (SF-12) also include elements of subjectivity and may suffer from selection and scale perception biases. Newer methods of nTOS evaluation, have introduced standardized criteria for diagnosis and analysis but remain largely reliant on subjective measures and lack objective testing to support a diagnosis of nTOS.

The subjectivity of current subjective methods of analysis of nTOS has resulted in diagnostic uncertainty, variability in treatment patterns, and inability to rigorously evaluate different modalities of treatment. Furthermore, the subjectivity of current methods of TOS analysis can reduce confidence in patients and physicians in the efficacy of current treatment methods.

Shortcomings mentioned here are only representative and are included simply to highlight that a need exists for improved detection and evaluation of TOS. Embodiments described herein address certain shortcomings but not necessarily each and every one described here or known in the art. Furthermore, embodiments described herein may present other benefits than, and be used in other applications than, those of the shortcomings described above.

### SUMMARY

Motion data recorded by wearable sensors may be used to detect and analyze TOS in a patient's upper extremity, such as a patient's arm. A patent may wear one or more sensors while performing a variety of exercises or while going about their daily life. The sensors may collect motion data from movement of one or both of the patient's arms and may transmit the motion data to a processing station for analysis. The processing station may analyze the data to determine one or more extremity performance parameters for one or both of the patient's arms. Based on the extremity performance parameters, the processing station may determine whether one or both of the patient's arms is subject to TOS. For example, erratic or limited motion profiles for an arm may indicate TOS. The processing station may also determine a severity of TOS in one or both of the patient's arms, based on the extremity performance parameters, and may recommend a TOS treatment, based on the extremity performance parameters. Examples of extremity performance parameters may include cardiac, arousal, cortisol level, or skin conductivity changes in response to a repetitive movement that exacerbates the symptoms of TOS or a digital biomarker indicative of at least one of slowness, weakness, exhaustion, rigidity, jerkiness, upper muscle strength, physiological parameters of pain, heart rate variability, cortisol level, or skin conductivity.

A data-based determination of whether an arm of a patient is subject to TOS may increase reliability in detection of TOS and may also improve patient outcomes. For example, data driven diagnosis and analysis of TOS may improve physician and patient confidence over prior subjective diagnosis methods, such as patient surveys. Extremity performance parameters can be compared against objective criteria to determine whether a patient's arm is subject to TOS. For example, motion data from patients may be input into a machine learning algorithm, along with survey data and other data regarding a patient's TOS status, and the machine learning algorithm may develop objective criteria, such as extremity performance parameters, for evaluating arm motion data for the presence of TOS. Furthermore, patient outcomes may be improved as motion data sets from previous patients who experienced positive treatment outcomes may be compared against motion data sets from current patients. For example, if an arm of a patient exhibits similar extremity performance parameters to those present in patients who respond well to a particular type of physical therapy, a physical therapy regimen may be suggested.

A system for detection and analysis of TOS may include a sensing device and a processing station. The sensing device may, for example, be wearable and may include at least one sensor configured to sense movement of an arm. The sensing device may also include a communications module coupled to the sensor and configured to transmit data from the sensing device and to the processing station. The communications module may, for example, include a wireless transmitter configured to transmit motion data wirelessly to the processing station. For example, motion data may be continuously transmitted to a processing station from the sensing device as a patient performs a series of exercises. Alternatively or additionally, the communications module may include a port for wired connection to the processing station. Example sensors that may be included in the sensing device include a uni-axial or tri-axial accelerometer, a gyroscope, and a heart rate monitor. The sensing device may also include a battery for powering the sensing device and a memory for storing sensed motion data. The sensing device may be attached to an arm of a patient. For example, the sensing device may be attached to an upper arm of a patient or to a lower arm of the patient. In some embodiments, multiple sensing devices may be attached to one or both arms of a patient. For example, first and second sensing devices may be attached to an upper right arm and a lower right arm of a patient. Likewise, third and fourth sensing devices may be attached to an upper left arm and a lower left arm of the patient. The processing station may be a server, a desktop, a laptop, a tablet, a mobile device, or other processing station.

Motion data regarding motion of an arm may be received from a sensor attached to an arm of a patient. A processing station may include a processor configured to receive and process such data. The processing station may include a communications module for communicating with the sensing device. For example, the processing station may communicate with the sensing device to receive motion data and to configure the sensing device. Motion data received by the processing station may include motion data gathered by a uni-axial or tri-axial accelerometer, a gyroscope, and/or a heart rate monitor of the sensing device.

Based on the received motion data, one or more extremity performance parameters for the arm may be determined. For example, a processor of the processing station may analyze motion data received from a sensing device to determine one or more extremity performance parameters for the arm to which the sensing device is attached. Example extremity performance parameters may include slowness of the arm, weakness of the arm, rigidity of the arm, and jerkiness of the arm. Slowness may, for example, include an average range of angular velocity of the sensing device, a duration between two consecutive zero crossing points during a movement of the sensing device, a rise time duration, and a fall time duration. Weakness may, for example, include a product of a range of angular acceleration of the sensing device and a range of angular deceleration. Rigidity may, for example, include a range of abduction rotation and adduction rotation. Jerkiness may, for example, include a highest frequency component of rotation. Slowness, weakness, rigidity, and jerkiness may, for example, be determined based on motion data recorded during a series of exercises for the arm, such as butterfly tests and/or press tests. Extremity performance parameters may also include a number of zero-crossing movements of the arm detected within a predetermined time period. For example, a patient may wear one or more sensors for a period of 24 hours, going about their normal unsupervised daily life, while data regarding zero crossings of one or both arms of the patient is recorded. Zero crossover points that do not satisfy a predetermined minimum time interval threshold may be discarded. In some embodiments, a moving average filter may be applied to data received from the sensing device in order to reduce artifacts in the data for more accurate extremity performance parameter determination. Thus, performance parameters may be determined based on motion data received from one or more sensing devices.

The extremity performance parameters may be indicative of whether the arm is subject to TOS, and a determination may be made of whether the arm is subject to TOS based on the extremity performance parameters. For example, a processor of a processing station may determine whether an arm of a patient is subject to TOS based on determined extremity performance parameters. An arm with slowness, weakness, rigidity, or jerkiness outside of a predetermined acceptable range may be subject to TOS. A severity of TOS in the arm may also be determined. For example, based on the extremity performance parameters, a score may be assigned to the arm from zero to one hundred, where zero indicates an asymptomatic arm and one hundred indicates an incapacitated arm. The more extremity performance parameters deviate from predetermined acceptable ranges for extremity performance parameters, the higher the score assigned to the arm.

A treatment plan may be selected based, at least in part, on the extremity performance parameters. For example a processor of a processing station may select a treatment plan based, at least in part, on the extremity performance parameters. Physical therapy or surgery to remedy TOS in the arm of the patient may be suggested. For example, extremity performance parameters may be compared against extremity performance parameters for previous patients who reacted positively to physical therapy. If the extremity performance parameters are similar to those of previous patients who reacted positively to physical therapy, a recommendation that the patient undergo physical therapy may be suggested. Other factors in addition to extremity performance parameters may also be considered, such as age, sex, BMI, and occupation.

The steps described herein may be included in code of a computer program product for execution by a computing device to carry out certain steps of the disclosure. The sensing device may communicate with the processing station through a wired connection or through a wireless communications protocol, such as Bluetooth or another wireless communications protocol, via wireless communications circuitry. Additional sensors may be used to monitor venous flow in connection with the requested motions. A heart rate sensor may also be used to monitor a heart rate and heart rate variability of a patient to evaluate physiological stress response as a surrogate of pain. Other sensors may be used to evaluate pain in response to a physical exercise such as respirator sensor to monitor changes in breathing rate because of pain, skin conduce sensor to measure physiological indicator pain in response to exercise, etc. To determine pain related to TOS condition, these sensors will measure changes in physiological response before and after an exercise that is designed to narrow the scalene triangle and provoke functional impairment of TOS.

As used herein the term “patient” refers to any person capable of experiencing TOS in one or more arms, according to any embodiment of the invention disclosed herein.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is an illustration of a sensing device and a processing station for detection and analysis of TOS according to some embodiments of the disclosure.

FIG. 2 is an illustration of a patient wearing multiple sensing devices, according to some embodiments of the disclosure.

FIG. 3 is an illustration of a patient wearing a sensing device in a butterfly test position according to some embodiments of the disclosure.

FIG. 4A is an illustration of a patient wearing a sensing device in a first press test position, according to some embodiments of the disclosure.

FIG. 4B is an illustration of a patient wearing a sensing device in a second press test position, according to some embodiments of the disclosure.

FIG. 5 is a graph of angular velocity of a patient arm unaffected by TOS during a TOS test according to some embodiments of the disclosure.

FIG. 6 is a graph of angular velocity of a patient arm subject to TOS during a TOS test according to some embodiments of the disclosure.

FIG. 7 is a flow chart of an example process for determining extremity performance parameters indicative of TOS according to some embodiments of the disclosure.

FIG. 8 is a graph of example arm speed during a TOS test according to some embodiments of the disclosure.

FIG. 9 is a graph of example arm power during a TOS test according to some embodiments of the disclosure.

FIG. 10 is a graph of example arm rising time during a TOS test according to some embodiments of the disclosure.

FIG. 11 is a graph of a mean arm speed during a TOS test compared with a DASH score for patients undergoing the TOS test according to some embodiments of the disclosure.

FIG. 12 is an illustration of an example patient wearing three sensing devices according to some embodiments of the disclosure.

FIG. 13 is illustration of example planes for zero-crossing of patient arms during TOS testing according to some embodiments of the disclosure.

FIG. 14 is a graph of arm speed of patient arms during TOS testing before and after treatment according to some embodiments of the disclosure.

FIG. 15 is a graph of a number of zero crossings of patient arms during TOS tests before and after treatment according to some embodiments of the disclosure.

FIG. 16 is an example method for detecting and analyzing TOS in a patient arm according to some embodiments of the disclosure.

FIG. 17 is for a graph illustrating diagnosis of TOS from non-TOS cases by measuring changes in digital markers post scalene muscle block according to embodiments of the disclosure.

### DETAILED DESCRIPTION

Patient motion data may be analyzed to detect thoracic outlet syndrome (TOS), such as nTOS, to determine a severity of TOS, and to suggest treatment options for TOS. One or more sensing devices may be attached to arms of a patient while the patient undergoes tests under observation and/or goes about their daily life unobserved. Motion data gathered by the sensing devices may be used to determine one or more extremity performance parameters of one or both of the patient's arms. TOS may be detected based on the extremity performance parameters. In some cases, a severity of TOS symptoms may be determined based on the extremity performance parameters, and a treatment plan may be proposed. Data-driven TOS detection and analysis may not only lead to improved physician and patient confidence in TOS diagnostics, but may also lead to enhanced patient outcomes through data-driven treatment suggestions based on past successful treatment of individuals with similar extremity performance parameters.

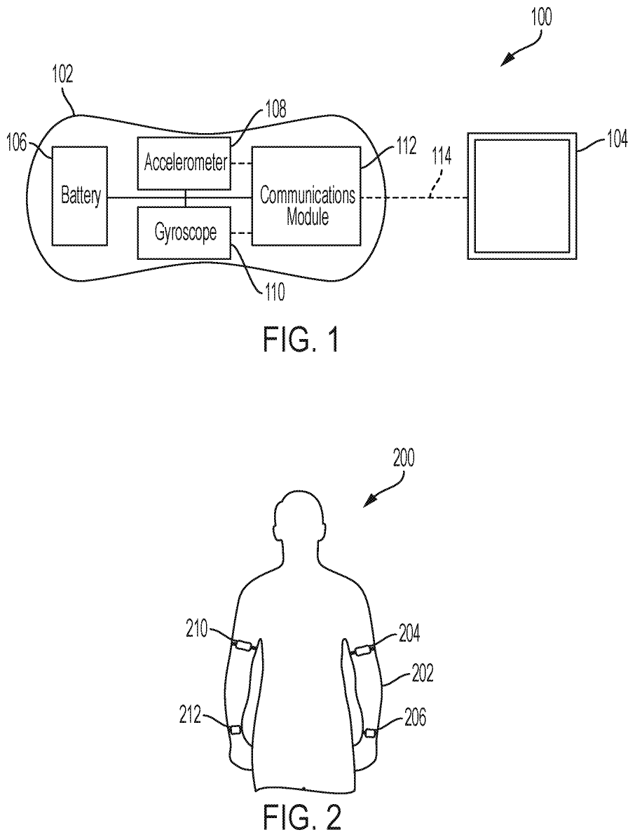

Motion data collected by a sensing device attached to an arm of a patient may be transmitted to a processing station for analysis. An example system 100 for collection and analysis of motion data for TOS diagnosis is shown in FIG. 1. A sensing device 102 may include one or more sensors for detection motion of an arm of a patient. For example, the sensing device 102 may be attached to an arm of a patient via adhesive, a strap, hooks and eyes, or other attachment mechanism. The sensing device 102 may include an accelerometer 108 for detecting acceleration of an arm of the a patient. The accelerometer 108 may be a uni-axial or tri-axial accelerometer for detecting acceleration and deceleration along three axes. The sensing device 102 may also include a gyroscope 110, such as a uni-axial or tri-axial gyroscope, for collecting rotational and directional motion data. The sensing device 102 may include a battery 106 for powering the internal components of the sensing device 102. The sensing device 102 may include a communications module 112 for communicating with a processing station 104. The communications module 112 may, for example, be a wireless communications module for communicating with the processing station 104 via a wireless connection such as a Bluetooth, Wi-Fi, cellular, or other wireless connection. For example, the sensing device 102 may be capable of continuous wireless transmission of measurements of accelerometer 108 and gyroscope 110 at a rate exceeding one hundred Hertz. In some embodiments, the communications module 112 may include a physical port for physically connecting to the processing station 104. The sensing device 102 may include a memory (not shown) for storing motion data sensed by the sensors 108, 110. For example, a sensing device 112 may be worn by a patient out of range of a wireless connection and may store motion data in a memory for retrieval by a technician at a later time. The sensing device 102 may further include additional sensors, such as heart rate and venous flow sensors. The sensing device 102 may be a lightweight and flexible medical-grade sensing device, to reduce artifacts that may be introduced by skin motion. The sensing device 102 may also be waterproof.

The sensing device 102 may connect to the processing station 104 via a connection 114. The connection 114 may be a connection over a wireless network, such as a Bluetooth connection or a connection over a local Wi-Fi network or cellular network, and/or a wired connection between the sensing device 102 and the processing station 104. In some embodiments the processing station 104 may be connected to the sensing device 102 to configure the sensing device 102. The processing station 104 may be a tablet, a laptop, a desktop, a server, a smart phone, or other computing platform capable of processing motion data. The processing station 104 may receive motion data from the sensing device 102 and may analyze the received motion data to detect TOS, such as nTOS. For example, the processing station 104 may process motion data to determine one or more extremity performance parameters for an arm of the patient and may determine whether the extremity performance parameters are indicative of nTOS, as described herein. The processing station 104 may, for example, extract 3D angles, 3D angular velocity, and 3D position parameters from the motion data received from the sensing device 102, and kinematic features of interest may be further derived from such features.

A patient may wear multiple sensing devices while the sensing devices gather motion data for one or both arms of the patient. FIG. 2 shows an example patient 200. An example patient 200 may have a left arm 202 with a first sensing device 204 coupled to the upper left arm and a second sensing device 206 coupled to the lower left arm. The patient 200 may have a right arm 208 with a third sensing device 210 coupled to the upper left arm and a fourth sensing device 212 coupled to the lower left arm. The sensing devices 204, 206, 210, 212 may collect motion data regarding movement of the right and left arms 208, 202 while the patient conducts arm movement exercises under observation and/or while the patient goes about their daily life unobserved. The sensing devices 204, 206, 210, 212 may be attached to the right and left arms 202, 208 via straps, as shown in FIG. 2, via adhesive, or via another attachment mechanism. The number of sensing devices worn by a patient may vary. For example, for collection of certain data sets a patient may wear a single sensing device, while collection of other data sets may require a patient to wear as many as five or more.

Sensing devices 204, 206, 210, 212 may be calibrated prior to collection of arm motion data during patient activity. For example, the sensing devices 204, 206, 210, 212 may be calibrated to remove a gravity component of measurements and to measure 3D joint angles of the patient 202 in reference to a fixed landmark. For example, the patient 202 may move a predefined distance, and sensor alignment estimates may be corrected based on data gathered during the movement. Axis correction may also be achieved when a patient rotates using quaternion algorithms. In some applications, such as when a patient is experience unilateral TOS, sensing devices on an arm not experiencing TOS, such as left arm sensing devices 204, 206, may be used as a control in analyzing data collected by sensing devices on the arm subject to TOS, such as right arm sensing devices 210, 212. In some embodiments, the system 200 may include a camera in place of or in addition to the use of one or more sensing devices or other means of motion tracking, to track and analyze patient motion and extremity performance parameters. Other devices may also be used to sense extremity performance parameters, such as kinetic and kinematic biomarkers. For example, upper muscle strength could be analyzed using a surface electromyography sensing device.

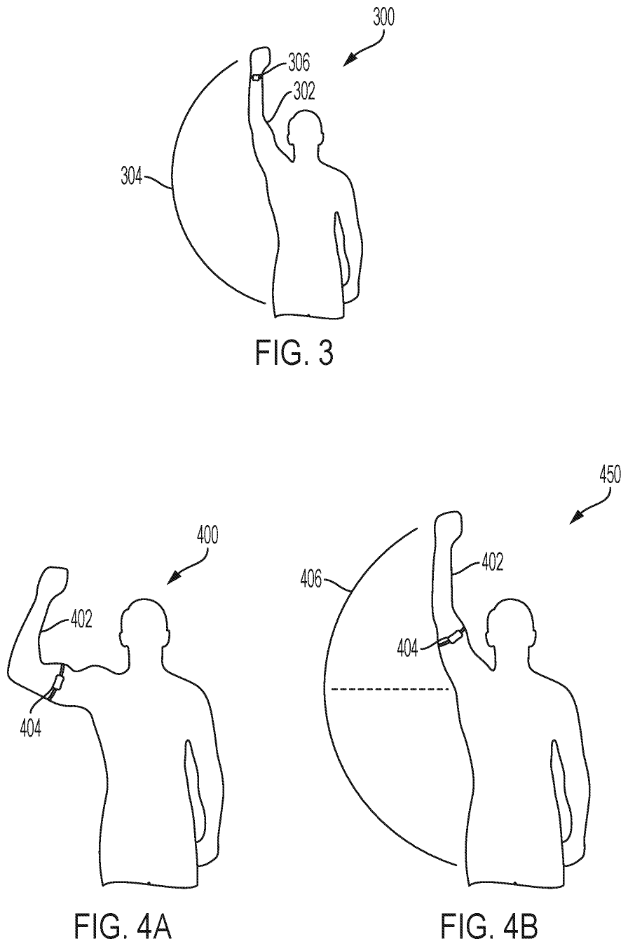

Motion data gathered while a patient performs a butterfly TOS test exercise may be useful in determining whether an arm of the patient is subject to TOS. The butterfly test may be based on the upper limb tension test (ULTT), a clinical test involving stretching of the brachial plexus to exacerbate the symptoms of nTOS. An example patient 300 performing a butterfly test is shown in FIG. 3. During a butterfly test, a patient may move an arm, such as a right arm 302 of the patient 300 from a first position against the side of the patient, as shown in FIG. 2, to a second position above the patient, as shown in FIG. 3. For example, the patient 300 may move the patient's right arm 302 along motion path 304. The patient 300 may fully extend the elbow of the right arm 302 while moving the right arm 302 along motion path 304, completing a one-hundred and eighty degree abduction upwards, and may then return the arm along motion path 304 to a resting position by the side of the patient 300. In some cases, the patient 300 may repeat the motion as rapidly as possible twenty times, or more. While performing the butterfly TOS test exercise, the patient may wear a sensing device 306 attached to a lower arm of the patient 300, such as to a wrist of the patient 300. A similar test may be performed on a left arm of a patient while the patient is wearing a sensing device on the lower left arm of the patient. The butterfly TOS test exercise may be performed in an arm of the patient that is not reported to be experiencing nTOS to establish an internal reference control, before performing the butterfly TOS test exercise in an arm of the patient that is reported to be experiencing nTOS. Motion data gathered during performance of butterfly TOS test exercises may be transmitted from sensing device 306 to a processing station for analysis.

A clinical test performed by a patient for TOS diagnosis may include movements designed to narrow the scalene triangle and provoke functional impairment of TOS. In some embodiments, the test may be performed before and after pharmacologically targeting anatomy specific to TOS, such as applying an anesthetic block of the anterior scalene muscle to relax its compression of the brachial plexus. For example, TOS may be diagnosed if a change in extremity performance parameters, such as kinetic and kinematic and physiological biomarkers, after pharmacologically targeting anatomy specific to TOS shows improvement greater than a pre-defined threshold. Such testing may also be used to quantify a severity of TOS based on a magnitude of extremity performance parameters, such as digital kinetic and kinematic biomarkers. Furthermore, additional sensors may be used to quantify changes in pain level before and after applying an anesthetic block of the anterior scalene muscle to improve diagnosis precision. These sensors could include cardia sensors, temperature sensor, skin conductivity sensor, cortisol measurement sensor or any sensor enables measuring physiological indicator of pain in response to the movements designed to narrow the scalene triangle and provoke functional impairment of TOS. In some applications, pain level is assessed by self-report before and after of the movements designed to narrow the scalene triangle and provoke functional impairment of TOS. Thus, in one embodiment of the disclosure, a method may include applying the diagnosis test, receiving motion data during the diagnosis test, determining extremity performance parameters, and determining whether the use is subject to TOS prior to treatment, pharmacologically targeting anatomy specific to TOS, and subsequently repeating the diagnosis test and associated reception and processing of data to determine TOS.

Motion data gathered while a patient performs a press TOS test exercise may also be useful in determining whether an arm of the patient is subject to TOS. The press test may be based on the upper limb tension test (ULTT), a clinical test involving stretching of the brachial plexus to exacerbate the symptoms of nTOS. An example patient 400 in a first position of a press TOS test exercise is shown in FIG. 4A. During a press test, a patient 400 may move an arm, such as a right arm 402 of the patient 400 from a first position with an elbow abducted at a ninety degree angle, as shown in patient 400 of FIG. 4A, to a second position above the patient, as shown in patient 450 of FIG. 4B. For example, the patient 400 may move the patient's right arm 402 along motion path 406. As shown in FIG. 4B, the patient 450 may fully extend the elbow of the right arm 402, completing a 180-degree abduction upwards, and may then return the arm 402 to the position of patient 400 of FIG. 4A with the elbow abducted ninety degrees. In some cases, the patient 400 may repeat the motion as rapidly as possible during a period of twenty seconds. While performing the press TOS test exercise, the patient may wear a sensing device 404 attached to an upper arm of the patient 400. A similar test may be performed on a left arm of a patient while the patient is wearing a sensing device on the upper left arm of the patient. The press TOS test exercise may be performed in an arm of the patient that is not reported to be experiencing nTOS to establish an internal reference control, before performing the press TOS test exercise in an arm of the patient that is reported to be experiencing nTOS. Motion data gathered during performance of press TOS test exercises may be transmitted from sensing device 404 to a processing station for analysis.

Other exercises, such as a rapid hand-over-head abduction task (the “Press Test”) hand-over abduction for a predefined duration (e.g., 20 seconds) that exacerbates the symptoms of nTOS by anatomically narrowing the scalene triangle with arm elevation may also be performed and monitored. For example, a patient may wear a sensing device on the upper arm and may repetitively perform hand-over-head exercise (e.g., for duration of 20 seconds) to exacerbate the symptoms of TOS by leveraging the anatomic narrowing of the scalene triangle that occurs with arm elevation. An angular velocity of the upper arm may be monitored throughout such a test. A zero-crossing technique may be used to identify the onset of the testing period. Real hand-over-head movements may be distinguished from noisy signals, in the collected motion data, by estimating an elapsed time between two consecutive detected zero-crossing points as an indicator of elevation duration, a range of angular velocity estimated between three consecutive zero-crossing points, and a magnitude of the maximum value of the angular velocity as an indicator of maximum speed of rotation during the flexion time. Valid zero-crossing points may be determined if each of the aforementioned parameters exceed a predefined threshold. Using the zero-crossing points, the maximum values for angular velocity during hand-over-head test may be recalculated. If any maximum value is less than twenty percent of the median value of all detected maximum angular velocity values, the zero-crossing points before and after that maximum value may be disregarded and/or removed. The first zero-crossing point may be considered the beginning of the test and the last zero-crossing point before the 20 second interval is complete may be considered the end of the test. Extremity performance parameters, such as biomarkers, including slowness, rigidity, exhaustion, and unsteadiness phenotype parameters listed in Table 1 below, may be extracted from the motion data, such as from analysis of zero-crossing points, and used in diagnosis of TOS. Furthermore coefficient of variance and percentage of decline may be calculated for each of the parameters listed in Table 1 below. Some dominant extremity performance parameters, such as biomarkers, that may be predictive of TOS may include a mean of abduction flexion time, as an indicator of slowness, a mean of elbow range of motion, as an indicator of rigidity, an inter-cycle variability of elbow extension time, as an indicator of a lack of extension steadiness, and a magnitude of decline in elbow rotation power after a 20 second rapid hand-over abduction-adduction test, as an indicator of exhaustion. In some embodiments, the sensor may be attached to wrist instead of upper arm and the test could be repetitive movements that stretches the brachial plexus to exacerbate the symptoms of TOS, called butterfly test. In butterfly test, the patient begins with the elbow fully extended and the arm completely adducted downwards (position 1). The upper extremity then completes 180 degree abduction upwards with the elbow remaining extending, reaching the “stick-up” position (position 2) and then returns to the starting position (position 1). The patient repeat this “jumping jack” cycle as rapidly as possible for a pre-defined period (e.g., 20 seconds). A single, body-worn sensor may collect sufficient data to determine such parameters. The use of a single sensor may reduce memory allocation and power cost for collection and analysis of extremity performance parameters. Use of a gyroscope in place of or in addition to an accelerometer may also enhance the clarity of the collected data.

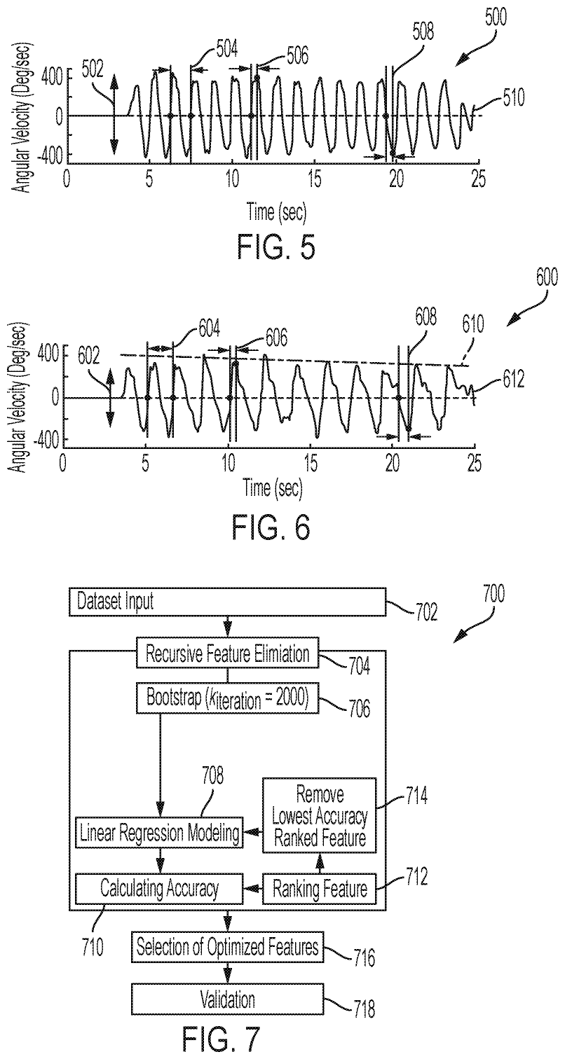

One example characteristic of arm movement that can be measured by a sensing device is angular velocity. For example, a sensing device may transmit motion data for an arm of a patient during a TOS test, such as the butterfly test exercise or the press test exercise, to a processing station, and the processing station may extract angular velocity for the arm of the patient from the motion data. An example graph 500 of angular velocity of a sensing device attached to a patient's lower arm during a butterfly test is shown in FIG. 5. Line 510 represents the angular velocity of an arm of a patient not experiencing TOS, in degrees per second, on the Y axis, over time, in seconds, on the X axis. A patient may perform twenty seconds of repetitions of a butterfly TOS test exercise, and data related to arm motion during the exercise, such as angular velocity 510, may be recorded. A wide range of motion characteristics may be determined based on angular velocity. For example, a speed of the arm may be determined based on the peak-to-peak amplitude 502 of the angular velocity. An abduction and adduction time 504 may be determined based on the time between first and third angular velocity zero crossover points. A rise time 506 of the arm may be determined based on the time between an amplitude zero crossover and a peak angular velocity. A fall time 508 may be determined based on the time between an amplitude zero crossover and a trough angular velocity. As shown in FIG. 5, the angular velocity 510 maintains a relatively consistent speed for the duration of the butterfly test exercise. The rise and drop times of the angular velocity 510 also remain relatively consistent throughout the duration of the exercise.

In some embodiments, the angular velocity 510 may be the angular velocity of a patient arm not experiencing TOS, while the other arm of the patient is experiencing TOS. The angular velocity data from the butterfly test of the arm not experiencing TOS may be collected as a baseline, against which to compare data from the arm that is experiencing TOS. In other embodiments, the angular velocity 510 of the asymptomatic arm may be a baseline angular velocity collected from a control group of healthy control subjects not experiencing TOS. The angular velocity 510 of the asymptomatic arm may be used as a baseline against which to compare angular velocity data from patients who may be suffering from TOS. If the angular velocity of a potential TOS patient performing a butterfly TOS test exercises exhibits characteristics similar to the angular velocity 510 of the asymptomatic arm, the patient may have a less severe case of TOS or may not be subject to TOS at all. If the angular velocity of the potential TOS patient performing butterfly TOS test exercises differs substantially from the angular velocity 510, for example, if the angular velocity of the potential TOS patient exhibits erratic movement with varying rise and fall times and a decreasing average speed, the patient's arm may be subject TOS.

An angular velocity for a TOS-affected arm performing a butterfly TOS test exercise can be compared against the angular velocity for an asymptomatic arm performing a butterfly TOS test exercise, as shown in FIG. 5. An example graph 600 of angular velocity of a sensor attached to a patient's lower arm during a butterfly test is shown in FIG. 6. Line 612 represents the angular velocity of a TOS-affected patient arm, in degrees per second, on the Y axis, over time, in seconds, on the X axis. The patient may perform twenty seconds of repetitions of a butterfly TOS test exercise while data related to arm motion during the exercise, such as angular velocity 612, is being recorded. A wide range of motion characteristics may be determined based on angular velocity. A speed of the arm may be determined based on the peak-to-peak amplitude 602 of the angular velocity. An abduction and adduction time 604 may be determined based on the time between first and third angular velocity zero crossover points. A rise time 606 of the arm may be determined based on the time between an amplitude zero crossover and a peak angular velocity. A fall time 608 may be determined based on the time between an amplitude zero crossover and a trough angular velocity. As shown in FIG. 6, the angular velocity 612 over time is somewhat erratic, with abduction and adduction time, rise time, and fall time, changing as the patient proceeds through a series of butterfly test exercise repetitions. Furthermore, as shown in FIG. 6, the average speed of the angular velocity, as shown by line 610, decreases over time. Varying rise and fall times, abduction and adduction times, and speed may be indicative of an arm subject to TOS. In some embodiments, the angular velocity 612 may be the angular velocity of a patient arm experiencing TOS, and may be compared against angular velocity of an asymptomatic arm of the patient, such as angular velocity 510 of FIG. 5. In other embodiments, the angular velocity 612 of the arm experiencing TOS may be compared against a baseline angular velocity collected from a control group of other patients not subject to TOS.

The angular velocity and/or other data collected during the movements may be analyzed to extract kinetic and kinematic biomarkers indicative of categories of slowness, weakness, rigidity, exhaustion, upper muscle strength, and unsteadiness. Extremity performance parameters may include such kinetic and kinematic biomarkers. Example measures that can be extracted from the data are shown in Table 1. Biomarkers may include objective, quantifiable, physiological and behavior data that are collected and measured by digital devices, such as wearables, cameras, and other devices. Digital biomarkers of upper extremity motor capacity may be particularly useful in diagnosing and selecting treatment for TOS. Additional kinetic or kinematic biomarkers can include mean, coefficient of variance, and percentage of decline of each of the measures of Table 1. The association of these extracted measures with characteristics is shown in Table 2.

TABLE 1

| Extracted measures | Example measurement |
|---|---|
| Angular velocity range | Range of angular velocity estimated |
| | by difference between maximum and |
| | minimum angular velocity peaks |
| Angle range | Range of abduction/adduction angle |
| Power range | Product of the angular velocity range |
| | and angular acceleration range |
| Rising time | Elapsed time to reach the maximum |
| | angular velocity during abduction |
| Falling time | Elapsed time to reach the minimum |
| | angular velocity during adduction |
| Rising + falling time | Sum of rising and falling times |
| Elbow abduction time | Duration of elbow abduction |
| Elbow adduction time | Duration of elbow adduction |
| Elbow abduction + | Sum of elbow abduction and |
| adduction time | adduction times |
| Elbow abduction/ | Number of elbow abduction/ |
| adduction rate | adduction per min |
| Number of abduction/ | Number of abduction/adduction |
| adduction | during test |

TABLE 2

| Upper extremity | Example | |
|---|---|---|
| characteristics | parameters | Example measurement |
| Slowness | Speed | Elbow angular velocity range |
| Slowness | Rise time | Duration of abduction |
| | | acceleration |
| Slowness | Fall time | Duration of adduction |
| | | acceleration |
| Slowness | Abduction time | Duration for rising arm from |
| | | the Position 1 to the Position 2 |
| Slowness | Adduction time | Duration for moving arm from |
| | | the Position 2 back the |
| | | Position 1 |
| Slowness | Abduction + | Total duration for a cycle of |
| | adduction time | abduction and adduction |
| Slowness | No. of abduction/ | Number of repetitions per 20 |
| | adduction | seconds |
| Weakness | Power | Product of the angular |
| | | acceleration rang and the |
| | | range of angular velocity |
| Rigidity | Range of motion | Range of abduction/adduction |
| | | rotation |
| Exhaustion | Decline in speed | Difference between the first |
| | | and last 10 seconds of angular |
| | | velocity |
| Exhaustion | Decline in power | Difference between the first |
| | | and last 10 seconds of power |
| Exhaustion | Increase in | Difference between the first |
| | abduction/ | and last 10 seconds of |
| | adduction time | abduction/adduction time |
| Exhaustion | Increase in | Difference between the first |
| | rise time | and last 10 seconds of rise |
| | | time duration |
| Unsteadiness | Speed variability | Coefficient of variation (CV) |
| | | of speed |
| Unsteadiness | Rise time | CV of rise time |
| | variability | |
| Unsteadiness | Abduction + | CV of abduction + adduction |
| | adduction | time |
| | variability | |
| Unsteadiness | Power variability | CV of power |
| Unsteadiness | Rigidity variability | CV of rigidity |

Biomarkers indicative of slowness may include speed (average range of angular velocity), duration of abduction+adduction, rise time (duration of abduction acceleration), fall time (duration of adduction acceleration), abduction time (duration from Position 1 to Position 2), adduction time (duration from Position 2 to Position 1), and total number of cycles. A weakness estimate may be computed as proportional to the product of range of angular velocity and range of angular acceleration. A rigidity estimate may be calculated as proportional to a range of abduction/adduction rotation calculated using quaternion and Kalman filters, as described. Each variable may be determined for each cycle of arm movement and the averages of the variables across multiple arm movement cycles may be compared between groups. Exhaustion may be determined as a decline in motor capacity (including speed, rise time, power) from the first and last ten-second interval. Unsteadiness may be quantified using a coefficient of variations for metrics indicative of slowness, power, and/or rigidity. 5-20 seconds, or more, of data regarding angular velocity may be used to estimate patient phenotypes (e.g., biomarkers) of interest and quantify patient exhaustion.

Motion data from TOS test exercises, such as the data illustrated in the graphs 500, 600 of FIGS. 5 and 6, may be used to determine a variety of extremity performance parameters that may be indicative of TOS. For example, zero crossover and peak detection algorithms may be applied to determine a variety of kinematics and kinematic features of arm movement from motion data, such as extremity performance parameters of slowness, weakness, rigidity, and jerkiness. Slowness may be indicated by an average range of angular velocity over duration of the butterfly test exercise, a duration between two consecutive zero-crossover points, such as abduction and adduction time 504, 604, rise time 506, 606, and fall time 508, 608. Weakness may be estimated based on power generated during abduction and adduction by multiplying a range of angular velocity by a range of angular acceleration, over the duration of the test. Rigidity may be determined by calculating a range of abduction and adduction rotation using quaternion and Kalman filters. Jerkiness may be determined based on the highest frequency rotation component of the exercise. Furthermore, mean values, standard deviation values, coefficient of variation values, and differences between the first and last ten seconds of shoulder abduction and adduction, which may indicate exhaustion, may be determined. A moving average filter, such as a six-point filter may be applied to recorded data, such as angular velocity 510, 612, to reduce artifacts with minimum reduction in magnitude of peak velocity. False detection may be minimized by excluding from analysis zero crossover points that do not satisfy minimum expected time-interval thresholds. Thus, using motion data, such as angular velocity captured during butterfly TOS test exercises, a variety of extremity performance parameters that indicate whether an arm of a patient is subject to TOS may be determined.

Machine learning algorithms may be applied to sets of motion data collected from arms subject to TOS and asymptomatic arms to determine extremity performance parameters that are indicative of TOS. An example method 700 for determining extremity performance parameters indicative of TOS is shown in FIG. 7. The method 700 may begin, at step 702, with input of multiple datasets of motion data. For example, multiple datasets of motion data for arms subject to TOS may be input, along with multiple datasets of motion data for asymptomatic arms. The motion data may include motion data from performing butterfly TOS test exercises and press TOS test exercises and motion data collected while patients are going about their daily routines. The motion data may include data from one or more uni-axial accelerometers, tri-axial accelerometers, uni-axial gyroscope, and/or tri-axial gyroscopes of sensing devices attached to one or both arms of patients.

The datasets may be passed, at step 704, to a recursive feature elimination algorithm. The recursive feature elimination algorithm may allow for selection of extremity performance parameters that are highly indicative of TOS, while allowing for elimination of extremity performance parameters that are not indicative of TOS. The recursive feature elimination algorithm may include bootstrapping, at step 706. The bootstrapping may include up to and exceeding 2000 iterations of random sampling and replacement of datasets for use in determination of extremity performance parameters that correlate closely with the presence of nTOS. Validation sets of input motion data may be selected during bootstrapping, at step 706, and passed to a validation process, at step 718. Training sets of input motion data may also be selected during bootstrapping, at step 706, and may be passed to a linear regression modeling stage, at step 708. DASH scores associated with the input data sets may also be input and may be used in linear regression modeling, at step 708, as a dependent variable to model sensor-derived output. Features of input motion data, such as extremity performance parameters, may be used as independent variables in the linear regression modeling of step 708. The linear regression modeling step 708 may feed into a calculating accuracy step 710. For example, accuracy of various extremity performance parameters at predicting TOS, when comparing parameters present in randomly selected motion datasets with input DASH scores for the datasets, may be determined. After accuracy is calculated at step 710, features, such as extremity performance parameters, may be ranked at step 712. For example, extremity performance parameters that correlate most closely to high DASH scores, indicating severe TOS, may be ranked above features that do not correlate to high DASH scores as closely. At step 714, the lowest accuracy ranked feature may be removed from analysis. Therefore, a feature that is not as indicative of TOS as other features may be removed. The steps of linear regression modeling, at step 708, calculating accuracy, at step 710, ranking features, at step 712, and removal of the lowest accuracy ranked feature, at step 714, may then repeat until a satisfactory set of extremity performance parameters is arrived at. Extremity performance parameter models arrived at using the machine learning algorithm of FIG. 7 may be adjusted by age, BMI, and sex. Other methods such as neural network, deep learning, and other artificial intelligent methods may be used to diagnose TOS and quantify its severity based on identified markers

At step 716, a number of optimized features may be selected based on the recursive feature elimination at step 704, including the linear regression modeling at step 708. For example, a number of extremity performance parameters that will produce the most reliable TOS prediction based on patient arm motion data may be selected. Thus, a set of extremity performance parameters for use in detection and analysis of TOS may be selected. The set of extremity performance parameters may also be used to provide a scale indicative of TOS severity, based on received arm motion data. At step 718, the results of the method 700 may be validated. For example, the set of extremity performance parameters may be adjusted for sensitivity, specificity, positive and negative predictive values, and area under curve. Validation sets of data selected during bootstrapping, at step 706, may be used to validate the selected extremity performance parameters. In some embodiments, data from a rapid elbow adduction-abduction test may be analyzed using the method 700. Demographics information, such as age, body mass index (BMI), and sex, may also be used as independent variables to improve the area under curve for distinguishing motion data from arms subject to TOS and motion data from asymptomatic arms. Thus, through a process of random sampling and replacement, a machine learning algorithm may enable validation of robustness and accuracy of a TOS diagnostic model by selecting some subsets of motion data for training and other subsets of motion data for validation in selecting a set of extremity performance parameters indicative of TOS.

A variety of methods may be used to compare motion datasets to determine extremity performance parameters. For example, one way analysis of covariance (ANCOVA), Fisher's exact tests, and Spearman's chi-square tests may be used to compare data between groups, such as comparing motion data of an arm of a patient subject to nTOS with motion data of the other arm of the patient not subject to nTOS, or comparing motion data from arms of individuals subject to nTOS with motion data from arms of individuals in a healthy control group. For example, an ANOVA model or McNemar test may be used to compare motion data of an arm of a patient subject to nTOS with motion data of the other arm of the patient not subject to nTOS to determine underlying correlation data of the same patient. Mann-Whitney U-tests may be used to compare between patients that respond to and patients that do not respond to physical therapy intervention. Pearson correlation coefficients or Spearman's chi-square test may be used to examine correlation between motion data received from sensing devices attached to patient arms and patient survey data, such as DASH or CBSQ data. For example, such methods may be used in the linear regression modeling at step 708 of FIG. 7. Sensitivity, specificity, accuracy, area under curve, and effect size may be calculated for motion data sets to evaluate model performance of the machine learning algorithm described with respect to FIG. 7 and to distinguish between affected and unaffected sides in an nTOS group, as well as to distinguish between patient and healthy control groups. Motion data may be evaluated with P<0.05 being considered statistically significant. Furthermore, Cohen's effect sizes may be analyzed to compare extremities of interest. For example, Cohen's effect sizes between 0.2 and 0.49 may be considered small, effect sizes between 0.5 and 0.79 may be considered medium, effect sizes between 0.8 and 1.29 may be considered large, and effect sizes of 1.3 or greater may be considered very large.

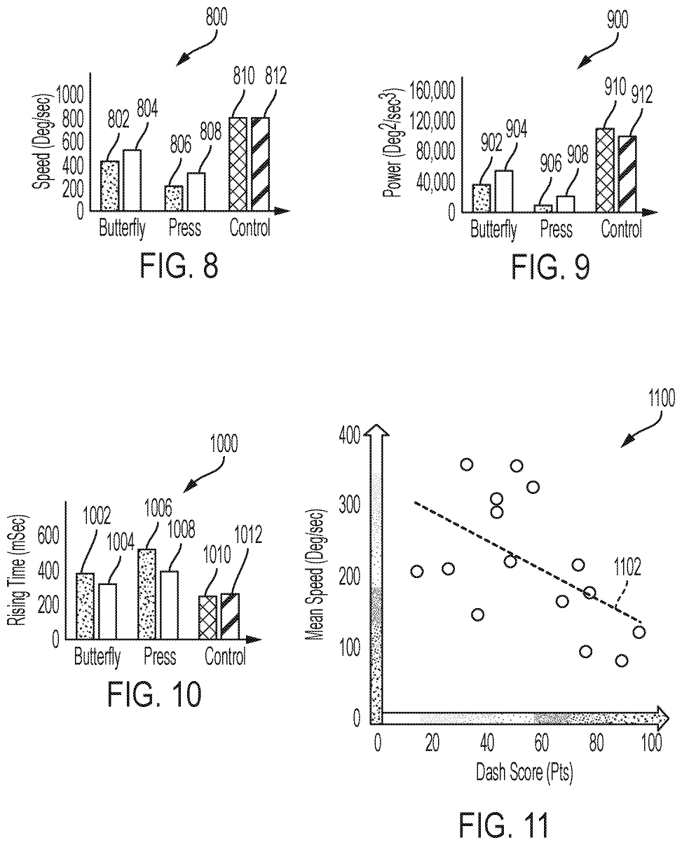

Speed, power, and rise time of arm movement during butterfly and press TOS test exercises may be analyzed to determine whether an arm is subject to TOS or asymptomatic. The bar graph 800 of FIG. 8 shows example average arm speed in degrees per second during butterfly and press exercises. In the test scenario from which the data of FIG. 8 was derived, eighteen patients diagnosed with nTOS were selected for testing having an average age of 37.2, an average BMI of 28.5, and an average DASH score of 55.3. The patients each had one arm affected by nTOS and one arm unaffected by nTOS. Sensors collected arm motion data, as described herein, during butterfly and press exercises performed by both arms affected by nTOS and arms not affected by nTOS in the patients. Line 802 represents an average speed of arms of patients affected by nTOS while performing butterfly TOS test exercises. Line 804 represents an average speed of arms of patients unaffected by nTOS while performing butterfly TOS test exercises. The Cohen's d between line 802 and line 804 was approximately 0.94, showing a large effect size. Line 806 represents an average speed of arms of patients affected by nTOS while performing press TOS test exercises. Line 808 represents an average speed of arms of patients unaffected by nTOS while performing press TOS test exercises. The Cohen's d between line 806 and line 808 was approximately 1.48, showing a large effect size. As shown in FIG. 8, the arms of patients that were unaffected by nTOS moved at a greater average speed than the arms of patients affected by nTOS, indicating that arm speed may be an effective extremity performance parameter in detecting nTOS. The differential between affected and unaffected arms for patients was greater in the press exercise than in the butterfly exercise.

A healthy benchmark was also established using motion data gathered from a group of ten healthy subjects, with an average age of 28.5, an average BMI of 28.5, and an average DASH score of 2.3. The healthy subjects performed at approximately the same speed for both butterfly and press exercises. Line 810 of FIG. 8, representing an average dominant arm speed of the healthy subjects, and line 812, representing an average non-dominant arm speed of the healthy subjects were almost identical, with a Cohen's d of 0.03. Furthermore, as shown in FIG. 8 the average speed of unaffected arms of patients during the butterfly and press tests, as shown by lines 804, 808, was lower than the average speed of the control group of healthy subjects, as shown by lines 810, 812, indicating that nTOS in one arm may negatively impact a patient's other arm.

FIG. 9 is a bar graph 900 of example average arm power in degrees squared per second cubed during butterfly and press exercises for the same group of test subjects described with respect to FIG. 8. Line 902 represents an average power of arms of patients affected by nTOS while performing butterfly TOS test exercises. Line 904 represents an average power of arms of patients unaffected by nTOS while performing butterfly TOS test exercises. The Cohen's d between line 902 and line 904 was approximately 0.9, showing a large effect size. Line 906 represents an average power of arms of patients affected by nTOS while performing press TOS test exercises. Line 908 represents an average power of arms of patients unaffected by nTOS while performing press TOS test exercises. The Cohen's d between line 906 and line 908 was approximately 1.01, showing a large effect size. As shown in FIG. 9, the arms of patients that were unaffected by nTOS moved with a greater average power than the arms of patients affected by nTOS, indicating that a lower arm movement power may be indicative of nTOS. As shown in FIG. 9, the differential between affected and unaffected arms for patients was slightly greater in the press exercise than in the butterfly exercise. A healthy benchmark was also established using the same group of healthy subjects described with respect to FIG. 8. The healthy subjects performed at approximately the same power for both butterfly and press exercises. Line 910, representing an average dominant arm power of the healthy subjects, and line 912, representing an average non-dominant arm power of the healthy subjects, were slightly different, with a Cohen's d of 0.21. Furthermore, as shown in FIG. 9 the average power of unaffected arms of patients during the butterfly and press tests, as shown by lines 904, 908, was lower than the average power of arms of the control group of healthy subjects, as shown by lines 910, 912, indicating that nTOS in an arm of a patient may negatively affect the patient's other arm as well.

The bar graph 1000 of FIG. 10 shows an example average arm rise time in milliseconds during butterfly and press exercises for the same group of test subjects described with respect to FIGS. 8 and 9. Line 1002 represents an average rise time for arms of patients affected by nTOS while performing butterfly TOS test exercises. Line 1004 represents an average rise time of arms of patients unaffected by nTOS while performing butterfly TOS test exercises. The Cohen's d between line 1002 and line 1004 was approximately 0.76, showing a large effect size. Line 1006 represents an average rise time of arms of patients affected by nTOS while performing press TOS test exercises. Line 1008 represents an average rise time of arms of patients unaffected by nTOS while performing press TOS test exercises. The Cohen's d between line 1006 and line 1008 was approximately 1.31, showing a large effect size. As shown in FIG. 10, the arms of patients that were affected by nTOS experienced a greater rise time than the arms of patients unaffected by nTOS, indicating that a high arm rise time may be indicative of nTOS. As shown in graph 1000, the differential between affected and unaffected arms of patients was slightly greater in the press exercise than in the butterfly exercise. A healthy benchmark was also established using the same group of healthy subjects described with respect to FIGS. 8 and 9. The healthy subjects performed at approximately the same rise time for both butterfly and press exercises. Line 1010, representing an average dominant arm rise time of the healthy subjects, and line 1012, representing an average non-dominant arm rise time of the healthy subjects were slightly different, with a Cohen's d of 0.21. Furthermore, as shown in FIG. 10 the average rise time of unaffected arms of patients during the butterfly and press tests, as shown by lines 1002-1008, was greater than the average rise time of the control group of healthy subjects, as shown by lines 1010, 1012, indicating that nTOS in an arm of a patient may negatively affect the patient's other arm.

To validate the sensor data analyzed in FIGS. 8-10, the nTOS patients were also asked to complete a DASH questionnaire. The DASH scores were then compared against an average speed for each of the patients, as shown in the graph 1100 of FIG. 11. Line 1102 represents the patient DASH score, on the X axis, plotted against patient arm speed, on the Y axis. As shown in FIG. 11, as the DASH score increases, indicating more severe nTOS symptoms, the mean speed, in degrees per second, decreases. There is a significant correlation between patient DASH scores and sensor-derived arm speed. Thus, arm speed is an effective extremity performance for detecting nTOS and determining a severity of nTOS. For example, in applying the machine learning algorithm described with respect to FIG. 7, average speed, variability of rise time, and variability of time of adduction were determined to distinguish affected and unaffected arms of nTOS patients, with a sensitivity and specificity of approximately 91.5% and 74.5% and an area under curve (AUC) of 83%. Furthermore, in applying the machine learning algorithm described with respect to FIG. 7, the sensitivity and specificity of average speed, variability of rise time, and variability of time of adduction in distinguishing between arms subject to nTOS and arms of healthy subjects were approximately 93.2% and 93.3%, with an AUC of 0.93. Thus, average speed, variability of rise time, and variability of time of adduction are highly correlated to the presence of TOS in a patient arm, and may be used as extremity performance parameters in determining whether a patient arm is subject to TOS. Thus, extremity performance parameters may be derived from motion data and may be used to determine whether an arm of a patient is subject to TOS and a severity of TOS symptoms of the arm.

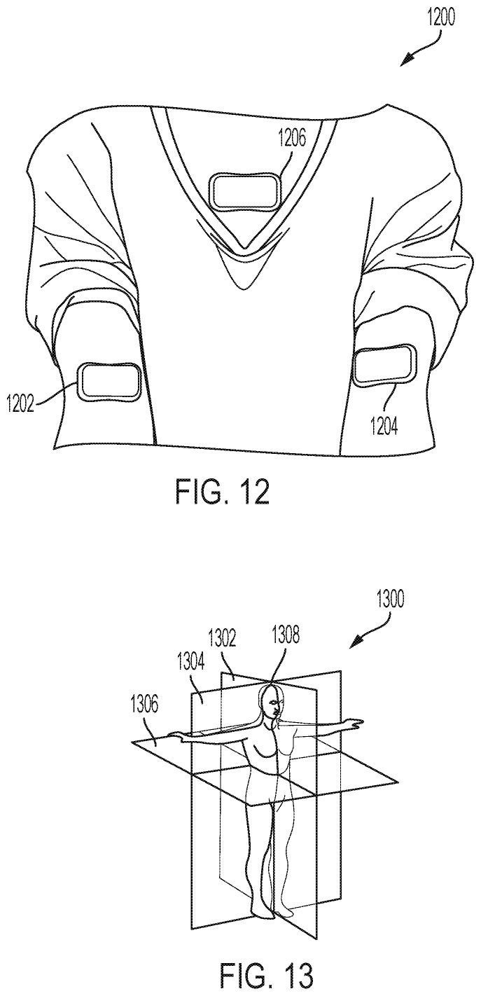

In addition to motion data gathered during TOS test exercises, motion data gathered while a patient goes about daily activities unobserved may be used to determine whether an arm of the patient is subject to TOS. Data regarding quality of sleep and heart rate variability may also be gathered, and may be useful in evaluating pain resulting from TOS. An example patient 1200 wearing a plurality of sensing devices is shown in FIG. 12. A first sensing device 1202 may be attached to a right arm, and a second sensing device 1204 may be attached to a left arm. In some cases the first and second sensing devices 1202, 1204 may be attached to an upper right arm and an upper left arm. A third sensing device 1206 may be attached to a torso of the patient 1200. For example, the third sensing device 1206 may be attached to an upper chest of the patient.

The chest sensing device 1206 may, for example, determine when the patient 1200 goes to sleep so that motion data from arm movements during sleep may be discarded. Motion data from the chest sensing device 1206 may be used to determine posture and physical activity of the patient 1200, such as when the patient 1200 is standing, sitting, lying, and walking. The arm sensing devices 1202, 1204 may record motion data from the arms while the patient 1200 goes about their daily activities. Motion data from the arm sensing devices 1202, 1204 may, for example, be used to determine a number of zero crossover movements of the arms of the patient 1200 during a twenty-four hour period. FIG. 13 is an example diagram 1300 of a variety of planes that intersect a patient 1308. For example, a sagittal plane 1302 may cross through from the front to the back of the patient 1308, perpendicular to a direction that the patient 1308 is facing. A transverse plane 1304 may extend outward from a waist of the patient 1308. A coronal plane 1304 may cross through the patient 1308, parallel to a direction the patient 1308 is facing. Motion data from sensing devices 1202, 1204 of FIG. 12 may be used to determine a number of times each arm of the patient crosses a transverse plane.

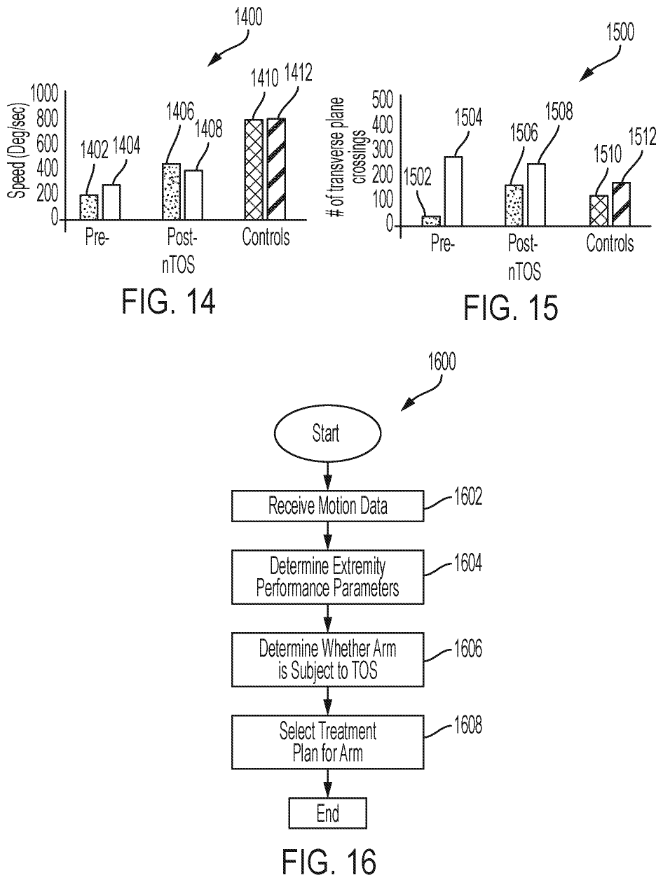

Extremity performance parameters such as an average arm speed and number of transverse plane crossings by an arm of a patient during an average day of use may be analyzed, along with speed, power, and rise time measured during butterfly and press exercises, to determine whether the arm is subject to TOS or asymptomatic. Furthermore, extremity performance parameters may be used to determine the effectiveness of treatments the patient has gone through, such as physical therapy and surgery. FIG. 14 is a bar graph 1400 of example average arm speed in degrees per second for patients before and after corrective surgery. For example, in the test scenario from which the data of FIG. 14 was derived, two patients diagnosed with nTOS were selected for testing, having an average age of 40, an average BMI of 29.5, and an average DASH score of 92.4. The patients each had one arm affected by nTOS and one arm unaffected by nTOS. Sensors collected arm motion data, as described herein, during butterfly and press exercises performed by both arms affected by nTOS and arms not affected by nTOS in the patients before and after surgery. Line 1402 represents an average speed of arms of patients affected by nTOS while performing test exercises under observation, prior to surgery. Line 1404 represents an average speed of arms of patients unaffected by nTOS while performing test exercises under observation, prior to surgery. Line 1406 represents an average speed of arms of patients affected by nTOS while performing TOS test exercises under observation after surgery. Line 1408 represents an average speed of arms of patients unaffected by nTOS while performing TOS test exercises under observation after surgery. As shown in FIG. 14, the arms of patients affected by TOS experienced a dramatic improvement in arm speed from arm speed before surgery, shown by line 1402, to arm speed after surgery, shown by line 1406. Arm speed in arms unaffected by nTOS also experienced improvement following surgery, as shown by line 1404 and line 1408. A healthy benchmark was also established using motion data gathered from a group of four healthy subjects, with an average age of 33.5, an average BMI of 24.1, and an average DASH score of 0.2. The healthy subjects performed at approximately the same speed for control dominant arms and control non-dominant arms. Line 1410, representing an average dominant arm speed of the healthy subjects, and line 1412, representing an average non-dominant arm speed of the healthy subjects were almost identical. As shown, surgery improved speed of the arms of nTOS patients, but did not increase speed to the levels of the healthy control group.

A number of transverse plane crossings for the same group of patients and healthy subjects described with respect to FIG. 14 was also determined. Patients wore upper arm sensing devices for a period of twenty-four hours, including a work period of approximately ten hours, going about their normal daily activities. Motion data recorded by the sensing devices was used to determine an average number of arm crossings of a transverse plane, over the twenty-four hour period of activity. The number of arm crossings of the transverse plane was determined by determining a number of upper arm zero-crossing points during vertical acceleration. A chest sensing device was also worn by patients and healthy subjects, and only transverse plane crossings while the patient was in the upright position were recorded. The number of arm crossings of the transverse plane was recorded for the patients before and after surgery. FIG. 15 is a bar graph 1500 of example average number transverse plane crossings of an arm during daily use. Line 1502 represents an average number transverse plane crossings of arms of patients affected by nTOS while going about daily activities unsupervised before surgery. Line 1504 represents an average number transverse plane crossings of arms of patients unaffected by nTOS while going about daily activities unsupervised before surgery. Line 1506 represents an average number transverse plane crossings of arms of patients affected by nTOS while going about daily activities unsupervised following surgery. Line 1508 represents an average number transverse plane crossings of arms of patients unaffected by nTOS while going about daily activities unsupervised following surgery. As shown in FIG. 15, the arms of patients that were affected by nTOS show a substantial increase in number of transverse plane crossings during unsupervised daily activity, from line 1502 before surgery to line 1506 after surgery. Furthermore, the average number of transverse plane crossings by arms of patients unaffected by nTOS decreased from line 1504 before surgery to line 1508 after surgery, possibly due to the surgery improving use of the arm subject to nTOS. A healthy benchmark was also established using the same group of healthy subjects described with respect to FIG. 14. The healthy subjects wore sensing devices on an upper dominant arm and an upper non-dominant arm while going about daily activities for 24 hours. Line 1510, representing an average number transverse plane crossings for a dominant arm of the healthy subjects during unsupervised daily use, and line 1512, representing an average number transverse plane crossings of a non-dominant arm of the healthy subjects during unsupervised daily use were recorded. As shown in FIG. 15, a number of transverse plane crossings of both nTOS subject arms and arms that were not subject to nTOS of patients, as shown by lines 1506 and lines 1508, were above the average number of transverse plane crossings for dominant and non-dominant arms, as shown by lines 1510 and 1512, of the healthy subjects. An increase in a number of transverse plane crossings during unsupervised daily use and average arm speed during supervised TOS test exercises following surgery may be indicative of a successful surgery.

Motion data from one or more sensing devices may be received and analyzed to detect and analyze TOS in a patient and, in some cases, to suggest a treatment for TOS. An example method 1600 for processing motion data to detect TOS is shown in FIG. 16. The method 1600 may begin with receiving motion data, at step 1602. Motion data may be received from sensing devices attached to a patient. For example, sensing devices may be attached to upper and lower arms of a patient and to a chest of a patient. The sensing devices may record and/or transmit data to a processing station while the patient engages in a variety of activities. For example, motion data may be recorded while a patient engages in TOS test exercises in a supervised or unsupervised environment, such as a butterfly TOS test exercises and press TOS test exercises. Motion data may also be recorded while a patient goes about their daily activities in an unsupervised environment, such as during a twenty-four hour transverse plane crossing test, as described herein. Motion data may be immediately transmitted from one or more sensing devices to a processing station as it is recorded, via a wireless connection such as a cellular, Wi-Fi, or Bluetooth connection. Alternatively, motion data may be recorded and stored in a memory of the sensing devices and may be transferred to a processing station at a later time via a wireless or wired connection.

At step 1604 extremity performance parameters may be determined based, at least in part, on the received motion data. For example, a processing station may receive motion data from one or more sensing devices and may analyze the motion data to determine one or more extremity performance parameters for the data. The extremity performance parameters for which the data is analyzed may, for example, be extremity performance parameters selected by the machine learning algorithm described with respect to FIG. 7. Extremity performance parameters may include slowness, weakness, rigidity, and jerkiness. Extremity performance parameters may further include a number of transverse plane crossings of an arm during a predetermined period of time, an average speed of an arm while performing TOS test exercises, a power of an arm while performing TOS test exercises, a rise time of an arm while performing TOS test exercises, and a fall time of an arm while performing TOS test exercises. Slowness may be indicated by an average range of angular velocity a series of exercises, a duration between two consecutive zero-crossover points, such as abduction and adduction time, rise time, and fall time. Weakness may be estimated based on power generated during abduction and adduction by multiplying a range of angular velocity and a range of angular acceleration, over the duration of the test. Rigidity may be determined by calculating a range of abduction and adduction rotation using quaternion and Kalman filters. Jerkiness may be determined based on the highest frequency rotation component of the exercise. Furthermore, mean values, standard deviation values, coefficient of variation values, and differences between the first and last ten seconds of shoulder abduction and adduction, which may indicate exhaustion, may be determined and may be used as extremity performance parameters. A moving average filter, such as a six point filter may be applied to motion data, such as angular velocity, to reduce artifacts with minimum reduction in magnitude of peak velocity. False detection may be minimized by excluding zero crossover points that do not satisfy minimum expected time-interval thresholds from analysis.

At step 1606, a determination may be made of whether an arm is subject to TOS. For example, a processing station may determine based on the determined extremity performance parameters whether an arm is subject to TOS, such as nTOS. If the arm is determined to be subject to TOS, a treatment plan may be determined, such as surgery or physical therapy. If the arm is determined not to be subject to TOS, a determination may be made that no treatment is required. For example, if extremity performance parameters for an arm are determined to be typical of arm motion of an arm subject to TOS, such as falling speed over a series of exercises, lengthy rise and fall times, or a low number of transverse plane crossings, a determination may be made that the arm is subject to TOS. In some cases, a score may be assigned to the arm based on the extremity performance parameters. For example, a score on a one hundred point scale may be assigned to the arm with zero indicating an asymptomatic arm and one hundred indicating a non-functional arm. The further extremity performance parameters deviate from a baseline of extremity performance parameters typical of a healthy arm, the higher the assigned score may be. In some cases the determination, including the score, may be compared against results of a DASH survey, a cervical brachial symptom questionnaire (CBSQ), a SF-12, a brief pain inventory (BPI), a pain catastrophizing scale (PCS) and/or a Zung self-rating depression scale (SDS) for the patient to verify the determination. The determination and extremity performance parameters may also be added to a database, for use in evaluation of future patients. The score or other determinations may be reported to the client through other means, such as a display, a monitor, a print-out, an email or text message, or a push notification.

At step 1608, a treatment for the arm may be selected. For example, the processing station may compare the determined extremity performance parameters with previous baselines of extremity performance parameters of patients who experienced positive results from certain treatments. For example, if an arm of a patient exhibits similar extremity performance parameters to parameters of arms of patients that, in the past, have experienced positive results following a certain physical therapy regimen, the physical therapy regimen may be recommended by the processing station as a possible treatment for the arm subject to TOS. If an arm of a patient exhibits similar extremity performance parameters to parameters of arms of patients that, in the past, have experienced positive results following a surgery, the surgery may be recommended by the processing station as a possible treatment for the arm subject to TOS. Furthermore, the processing station may perform statistical analysis of past outcomes and may provide a probability of success of a variety of possible treatment methods. Factors considered in selecting a treatment for the arm may also include age, sex, BMI, a comorbidity index, cognitive performance, depression, participation in competitive athletics, a length of duration of symptoms, chronic pain conditions such as fibromyalgia, preoperative opioid use, preoperative extremity neurologic deficits, complications of surgery, coverage under a worker's compensation insurance policy, participation in heavy manual labor, marriage status, and education level. For example, a machine learning model similar to the method described with respect to FIG. 7 may be applied to outcome data to determine one or more treatment outcome predictive factors, which may include extremity performance parameters, to use in selecting the treatment for the arm. In some cases, detected extremity performance parameters, such as kinetic and kinematic and physiological biomarkers, may be used to predict responsiveness of a patient to conservative therapies, such as physical therapy, electrical stimulation, and other non-surgical intervention. The prediction of responsiveness may, for example, be based on a magnitude of extremity performance parameters or on a change in extremity performance parameters following pharmacological targeting of anatomy specific to TOS. A response of a patient to therapy, such as surgery, physical therapy, or other TOS therapy, may be tracked by sensing and analyzing extremity performance parameters throughout and/or following such therapy, such as by comparing various extremity performance parameters measured before therapy with extremity performance parameters measured after therapy. Thus, motion data may be used to determine extremity performance parameters, and the extremity performance parameters may be used to determine whether an arm is subject to TOS, a severity of TOS symptoms of the arm, and a possible treatment for TOS in the arm.

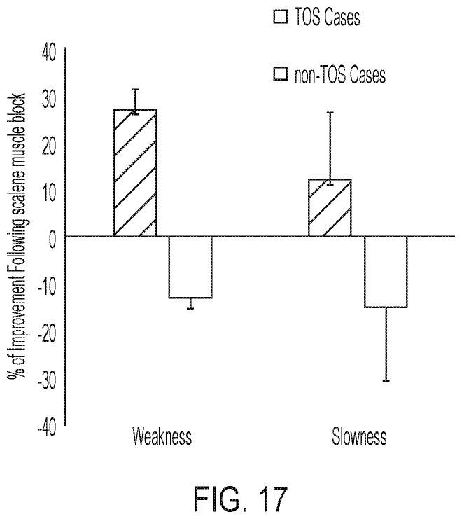

In some cases, detected extremity performance parameters, such as kinetic and kinematic and physiological biomarkers, may be used for diagnosis of TOS cases from non-TOS cases presenting with signs and symptoms compatible of TOS. The distinguishing of TOS cases from non-TOS cases with overlapping symptoms (e.g., radiculopathy, shoulder injury, ulnar nerve entrapment, etc.), for example, may be based on measuring a magnitude of extremity performance parameters or on a change in extremity digital markers following pharmacological targeting of anatomy specific to TOS. FIG. 17 illustrates slowness and weakness digital markers extracted from the press test before and after blocking scalene muscle for a group of patients with TOS condition and a group of patients without TOS, but with similar symptoms, which redistricts extremity performance (e.g., shoulder pain). The graph of FIG. 17 illustrates that the two groups can be distinguished using this technique.

While the sensing and data analysis apparatus, systems, and methods disclosed herein is described with respect to detection, analysis, and treatment of nTOS, the disclosed apparatus, system, and methods may also be used in detection, analysis, and treatment of other conditions. For example, the apparatus, systems, and methods disclosed herein may be applied to detection, analysis, and treatment of cervical radiculopathy, shoulder injury, regional pain syndrome, and other nerve compression syndromes such as ulnar entrapment and carpal tunnel syndrome.

The schematic flow chart diagram of FIG. 16 is generally set forth as a logical flow chart diagram. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The operations described above as performed by a controller may be performed by any circuit configured to perform the described operations. Such a circuit may be an integrated circuit (IC) constructed on a semiconductor substrate and include logic circuitry, such as transistors configured as logic gates, and memory circuitry, such as transistors and capacitors configured as dynamic random access memory (DRAM), electronically programmable read-only memory (EPROM), or other memory devices. The logic circuitry may be configured through hard-wire connections or through programming by instructions contained in firmware. Further, the logic circuitry may be configured as a general-purpose processor capable of executing instructions contained in software. If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method, comprising:
receiving, from a motion tracking device, motion data regarding user motion during a diagnosis test;
determining, based at least in part on the received motion data, one or more extremity performance parameters; and
determining, based at least in part on the one or more extremity performance parameters, whether the user is subject to thoracic outlet syndrome (TOS).

2. The method of claim 1, wherein the one or more extremity performance parameters comprises at least one of cardiac, arousal, cortisol level, or skin conductivity changes in response to a repetitive movement that exacerbates the symptoms of TOS or a digital biomarker indicative of at least one of slowness, weakness, exhaustion, rigidity, jerkiness, upper muscle strength, physiological parameters of pain, heart rate variability, cortisol level, or skin conductivity.

3. The method of claim 1, wherein the motion tracking device comprises at least one of a uni-axial gyroscope or a uni-axial accelerometer.

4. The method of claim 1, wherein the one or more extremity performance parameter comprises a measure of repetitive movement of user's arm within a predetermined time period that exacerbates the symptoms of TOS, wherein the repetitive movement of user's arm comprises movements that narrow the scalene muscle triangle.

5. The method of claim 1, wherein determining whether the user is subject to TOS is based, at least in part, on changes greater than a pre-defined threshold in the one or more extremity performance parameters from pre- to post-pharmacologically targeting anatomy specific to TOS.

6. The method of claim 1, further comprising selecting a TOS treatment plan for the arm based, at least in part, on the extremity performance parameters, when the arm is determined to be subject to TOS.

7. The method of claim 1, wherein determining one or more extremity performance parameters for the arm comprises discarding zero crossover points that do not satisfy a predetermined minimum time interval threshold.

8. The method of claim 1, wherein determining, based at least in part on the extremity performance parameters, whether the arm is subject to thoracic outlet syndrome (TOS) comprises assigning a score to the arm, based at least in part on the extremity performance parameters, wherein the score indicates a range from an asymptomatic arm to an incapacitated arm.

9. A system, comprising:
a processing station, comprising a processor configured to perform steps comprising:
receiving the motion data regarding motion of the arm from the sensing device;
determining, based at least in part on the received motion data, one or more extremity performance parameters for the arm; and
determining, based at least in part on the extremity performance parameters, whether the arm is subject to thoracic outlet syndrome (TOS).

10. The system of claim 9, further comprising: a sensing device, comprising:
a sensor configured to sense movement of the arm; and
a communications module coupled to the sensor,
wherein the communications module is configured to transmit motion data regarding movement of the arm sensed by the sensor to the processing station for extremity performance analysis; and

11. The system of claim 9, wherein the sensor comprises at least one of a uni-axial gyroscope, a uni-axial accelerometer, or a camera.

12. The system of claim 9, wherein the extremity performance parameter comprises a number of zero-crossing movements within a predetermined time period to exacerbate the symptoms of TOS.

13. The system of claim 9, further comprising selecting a TOS treatment plan for the arm based, at least in part, on the extremity performance parameters when the arm is determined to be subject to TOS.

14. The system of claim 9, wherein determining one or more extremity performance parameters for the arm comprises applying a moving average filter to the received motion data to reduce artifacts.

15. The system of claim 9, wherein determining one or more extremity performance parameters for the arm comprises discarding zero crossover points that do not satisfy a predetermined minimum time interval threshold.

16. The system of claim 9, wherein determining, based at least in part on the extremity performance parameters, whether the arm is subject to thoracic outlet syndrome (TOS) comprises assigning a score from zero to one-hundred to the arm, wherein a score of zero indicates an asymptomatic arm and a score of one hundred indicates an incapacitated arm.

17. A computer program product comprising:
a non-transitory computer readable medium comprising instructions to perform steps comprising:
receiving, from a sensing device attached to an arm of a patient, motion data regarding motion of the arm;
determining, based on the received motion data, one or more extremity performance parameters for the arm; and
determining, based at least in part on the extremity performance parameters, whether the arm is subject to thoracic outlet syndrome (TOS).

18. The computer program product of claim 17, wherein the extremity performance parameter comprises a number of zero-crossing movements within a predetermined time period, and wherein determining one or more extremity performance parameters for the arm comprises discarding zero crossover points that do not satisfy a predetermined minimum time interval threshold.

19. The computer program product of claim 17, wherein the computer program product further comprises instructions to perform steps comprising selecting a TOS treatment plan for the arm based, at least in part, on the extremity performance parameters, when the arm is determined to be subject to TOS.

20. The computer program product of claim 15, wherein determining, based at least in part on the extremity performance parameters, whether the arm is subject to thoracic outlet syndrome (TOS) comprises determining a score that indicates a range from an asymptomatic arm to an incapacitated arm.
